(19) United States
(12) Patent Application Publication
(10) Pub. No.: US 2025/0359851 A1
(43) Pub. Date: Nov. 27, 2025

(54) ULTRASONIC IMAGE DIAGNOSTIC SYSTEM, ULTRASONIC IMAGE DIAGNOSTIC PROGRAM, ULTRASONIC DIAGNOSTIC APPARATUS, AND ULTRASONIC IMAGE DIAGNOSTIC METHOD

(71) Applicant: KONICA MINOLTA, INC., Tokyo (JP)

(72) Inventors: Hiroshi Imamura, Tokyo (JP); Takashi Sakai, Yokohama-shi (JP)

(73) Assignee: KONICA MINOLTA, INC., Tokyo (JP)

(21) Appl. No.: 19/214,816

(22) Filed: May 21, 2025

(30) Foreign Application Priority Data
May 22, 2024 (JP) 2024-083690

(51) Int. Cl. A61B 8/00

(52) U.S. Cl. CPC A61B 8/463; A61B 8/467; A61B 8/54; A61B 8/565

(57) ABSTRACT

An ultrasonic image diagnostic system which includes: an ultrasonic diagnostic apparatus which is adapted to generate a first ultrasonic image based on a first ultrasonic image data obtained by transmitting and receiving ultrasonic signals using an ultrasonic probe, wherein the ultrasonic diagnostic apparatus has a display part for displaying the first ultrasonic image, and an information terminal having an input part and a display part, wherein the ultrasonic diagnostic apparatus and the information terminal are connected via a network, wherein the ultrasonic diagnostic apparatus generates a second ultrasonic image from a second ultrasonic image data different from the first ultrasonic image data in response to an instruction sent from the input part of the information terminal to the ultrasonic diagnostic apparatus, and the display part of the information terminal is adapted to display the second ultrasonic image independently of the first ultrasonic image displayed on the ultrasonic diagnostic apparatus.

### CROSS REFERENCE TO THE RELATED APPLICATION

The entire disclosure of Japanese patent Application No. 2024-083690, filed on May 22, 2024, is incorporated herein by reference in its entirety.

### BACKGROUND

### 1. Technological Field

The present invention relates to an ultrasonic image diagnostic system, an ultrasonic image diagnostic program, an ultrasonic diagnostic apparatus, and an ultrasonic image diagnostic method for remotely displaying an ultrasonic image.

### 2. Description of the Related Art

Conventionally, many medical diagnoses based on ultrasonic images of organs and tissues of living tissues have been performed. An ultrasonic diagnostic apparatus that generates an ultrasonic image transmits an ultrasonic to a living body, receives an echo reflected by an organ or a tissue, and images the echo as a cross-sectional image.

In recent years, a usage form in which an ultrasonic diagnostic apparatus and a mobile terminal are remotely connected to each other via a network and an imaging person of the ultrasonic diagnostic apparatus and a medical specialist using the mobile terminal share an ultrasonic image is becoming widespread.

For example, Japanese Translation of PCT International Publication 2020-508195 discloses an ultrasonic imaging system in which an ultrasonic image is shared between a pocket echo device and an image workstation. Specifically, it is described that the pocket echo device receives, from the image workstation side, a permission/non-permission command signal related to transmission and reception of an ultrasonic signal in the probe and a permission/non-permission command signal related to display of an ultrasonic image. Then, it is described that an operation command for the pocket echo device is transmitted from the image workstation.

Furthermore, Japanese Unexamined Patent Publication No. 2023-039624 discloses sharing an ultrasonic image, a camera image, a chat function, and a touch command screen between an ultrasonic diagnostic apparatus that is a server and an ultrasonic diagnostic apparatus that is a client. Then, Japanese Unexamined Patent Publication No. 2023-039624 discloses that a mouse pointer is displayed at an arbitrary position on a screen of a server from a client side, and an operation instruction is given to the server side from a client side to which priority is given among a plurality of clients.

### SUMMARY

In the ultrasonic diagnostic apparatus and the mobile terminal according to the above-described conventional technology, both an operator of the ultrasonic diagnostic apparatus and an operator of the mobile terminal at a remote location may be able to perform a screen display operation. In this situation, for example, when a doctor on duty or an emergency doctor who operates the ultrasonic diagnostic apparatus performs a treatment such as an urgent inspection or echo-guided puncturing, the doctor or the emergency doctor may want to continue to display a screen including an ultrasonic image currently observed by the doctor or the emergency doctor without changing the layout. That is, the user may not want the display screen to be changed. On the other hand, a healthcare professional such as a medical specialist at a remote site may want to display a screen different from that on the ultrasonic diagnostic apparatus side due to a difference in his/her role, observation purpose, or display operation environment.

That is, this is a case where the ultrasonic diagnostic apparatus and the mobile terminal want to change the display processing unique to ultrasonic for a predetermined reason. For example, it is necessary to obtain additional information necessary for advice or accurate diagnosis, or it is necessary to preferentially or limitedly display more important information because the resolution of the display part of the information terminal is smaller than that of the display part of the ultrasonic diagnostic apparatus.

An example of the former includes a case where, in order that a medical specialist at a remote site obtains advice or information necessary for accurate diagnosis, (i) an ultrasonic image for diagnosis currently synchronously displayed is frozen at a specific timing, and the shape of a tissue or a blood vessel is measured on the still image to display the image and the measurement result, and (ii) an ultrasonic image (blood flow image/morphology image) in a mode different from that of the ultrasonic image (morphology/blood flow image) currently synchronously displayed is live-displayed.

As an example of the latter, in a case where a normal B-mode image and a B-mode image on which nerve enhancement data is superimposed are displayed side by side on the display part of the ultrasonic diagnostic apparatus, the B-mode image on which nerve enhancement data is superimposed is preferentially displayed (on the front side or in a large size) on the display part of the low-resolution information terminal. Alternatively, there is a case where only the B-mode image on which the nerve enhancement data is superimposed is displayed.

An object of the present invention is to provide an ultrasonic image diagnostic system that causes an image being displayed on an ultrasonic diagnostic apparatus side, an image being displayed on an information terminal side, and an image obtained by giving a remote instruction to the apparatus and being displayed on the information terminal side to be displayed independently of each other.

To achieve at least one of the abovementioned objects, according to an aspect of the present invention, the followings are provided.
(1) An ultrasonic image diagnostic system includes: an ultrasonic diagnostic apparatus which is adapted to generate a first ultrasonic image based on a first ultrasonic image data obtained by transmitting and receiving ultrasonic signals using an ultrasonic probe, wherein the ultrasonic diagnostic apparatus has a display part for displaying the first ultrasonic image, and an information terminal having an input part and a display part, wherein the ultrasonic diagnostic apparatus and the information terminal are connected via a network, wherein the ultrasonic diagnostic apparatus generates a second ultrasonic image from a second ultrasonic image data different from the first ultrasonic image data in response to an instruction sent from the input part of the information terminal to the ultrasonic diagnostic apparatus, and the display part of the information terminal is adapted to display the second ultrasonic image independently of the first ultrasonic image displayed on the ultrasonic diagnostic apparatus.
(2) The ultrasonic image diagnostic system according to (1), wherein the display part of the information terminal changes from a state in which the first ultrasonic image is displayed to a state in which the second ultrasonic image is independently displayed in response to the instruction.
(3) The ultrasonic image diagnostic system according to (2), wherein the information terminal includes an instruction unit which is adapted to give an instruction from the input part to the ultrasonic diagnostic apparatus, and wherein the instruction unit is an independent display instruction unit which is adapted to allow the display part of the information terminal to independently display the second ultrasonic image.
(4) The ultrasonic diagnostic apparatus according to (2), wherein the ultrasonic diagnostic apparatus includes an independent display instruction unit for instructing the display part of the information terminal to independently display the second ultrasonic image.
(5) The ultrasonic image diagnostic system according to (1) or (2), wherein the display part of the information terminal is adapted to change from a state in which the second ultrasonic image is independently displayed to a state in which the first ultrasonic image is displayed.
(6) The ultrasonic image diagnostic system according to (5), wherein the display part of the information terminal includes an independent display cancelling unit that is adapted to allow the display part of the information terminal to change from the state in which the second ultrasonic image is independently displayed to the state in which the first ultrasonic image is displayed.
(7) The ultrasonic diagnostic apparatus according to any one of (1) to (4), wherein the instruction is an instruction for the ultrasonic diagnostic apparatus to specify a predetermined mode or preset.
(8) A computer readable nonvolatile storage medium storing a computer program, the computer program, when executed by a processor of an ultrasonic diagnostic apparatus including an ultrasonic probe and a display part, causing the processor to perform: generating first ultrasonic image data from an ultrasonic signal transmitted and received using the ultrasonic probe; displaying a first ultrasonic image based on the first ultrasonic image data on the display part; transmitting the first ultrasonic image data to an information processing apparatus; independently generating second ultrasonic image data different from the first ultrasonic image data based on an instruction from the information processing apparatus; and transmitting the second ultrasonic image data to the information processing apparatus.
(9) A computer readable nonvolatile storage medium storing a computer program, the computer program, when executed by a processor of an information processing apparatus including an input section and a display part, causing the processor to perform: receiving first ultrasonic image data from an ultrasonic diagnostic apparatus which displays a first ultrasonic image; displaying a first ultrasonic image based on the first ultrasonic image data on the display part; instructing the ultrasonic diagnostic apparatus by an input from the input section; receiving second ultrasonic image data different from the first ultrasonic image data from the ultrasonic diagnostic apparatus; and displaying a second ultrasonic image based on the second ultrasonic image data on the display part.
(10) An ultrasonic diagnostic apparatus comprising: an image data generation section generating first ultrasonic image data and/or second ultrasonic image data on the basis of ultrasonic signals transmitted and received by using an ultrasonic probe; a display part displaying an ultrasonic image generated on the basis of the first ultrasonic image data or the second ultrasonic image data; and a communication section transmitting the first ultrasonic image data or the second ultrasonic image data to an information processing apparatus and receiving an instruction from the information processing apparatus, wherein the image data generation section independently generates the second ultrasonic image data different from the first ultrasonic image data according to the instruction accepted by the communication section when the first ultrasonic image is displayed on the display part, and the communication section transmits the second ultrasonic image data to the information processing apparatus.
(11) A computer readable nonvolatile storage medium storing a computer program, the computer program, when executed by a processor of an information processing apparatus including an input section and a display part, causing the processor to perform: receiving first ultrasonic image data from an ultrasonic diagnostic apparatus which displays a first ultrasonic image; displaying a first ultrasonic image based on the first ultrasonic image data on the display part; instructing the ultrasonic diagnostic apparatus by an input from the input section; receiving second ultrasonic image data different from the first ultrasonic image data from the ultrasonic diagnostic apparatus; and displaying a second ultrasonic image based on the second ultrasonic image data on the display part.

According to the present invention, it is possible to provide an ultrasonic diagnostic apparatus system in which an image being displayed on an ultrasonic diagnostic apparatus side, an image being displayed on an information terminal side, and an image obtained by giving a remote instruction to the apparatus and being displayed on the information terminal side are independently displayed, and thus it is possible to improve user convenience.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a diagram illustrating a configuration of an ultrasonic image diagnostic system of an embodiment.

FIG. 2 is a flowchart for explaining the operation of the ultrasonic image diagnostic system.

FIG. 3 is a diagram illustrating the contents of ultrasonic inspection in example 1 and example 2.

FIG. 4A is a diagram illustrating display content of a display part of the ultrasonic diagnostic apparatus.

FIG. 4B is a view illustrating the display contents of the display part of the information terminals. 5A is a diagram illustrating an ultrasonic image of the main artery on the display part of the ultrasonic diagnostic apparatus.

FIG. 5B is a diagram illustrating an ultrasonic image of the main artery on a display part of an information device.

FIG. 5C is a diagram illustrating an ultrasonic image of the display part of the information device after the operation instruction.

FIG. 6 is a diagram illustrating details of a remote control floating window.

FIG. 7 is a view illustrating an example in which operation buttons for setting an independent display instruction function and an independent display cancellation instruction function are provided in the ultrasonic diagnostic apparatus.

FIG. 8 is a view illustrating an example in which an ultrasonic image to be independently displayed is overwritten and displayed.

FIG. 9 is a view illustrating an example in which a first ultrasonic image and a second ultrasonic image are displayed in different windows.

FIG. 10 is a view illustrating an example in which a first ultrasonic image and a second ultrasonic image are displayed on different tabs of the same window.

FIG. 11 is a view illustrating an example of marking an ultrasonic image by using a drawing tool.

FIG. 12 is a flowchart for explaining the details of the independent display processing.

FIG. 13A is a diagram illustrating a color Doppler image of a long-axis cross section of the left sternal edge on the display part of the ultrasonic diagnostic apparatus.

FIG. 13B is a diagram illustrating a color Doppler image of a long-axis cross-section of the left margin of the sternum on the display part.

FIG. 13C is a diagram illustrating a B-mode image of a long-axis cross section of the left margin of the sternum after an independent display instruction on the display part of the information device.

FIG. 14A is a view illustrating a B-mode image on which emphasis information concerning puncture needles, nerves, and blood vessels in the femoral region is superimposed on the display part of the ultrasonic diagnostic apparatus.

FIG. 14B is a diagram illustrating a B-mode image on which highlighting data related to puncture needles, nerves, and blood vessels in the femoral region is superimposed on the display part.

FIG. 14C is a diagram illustrating an example in which a B-mode image on which any emphasis date related to puncture needles, nerves, and blood vessels in the femoral region is not superimposed is displayed on the display part after an independent display instruction.

FIG. 15A is a diagram illustrating a B-mode image of a femoral region on a display part of an ultrasonic diagnostic apparatus.

FIG. 15B is a diagram illustrating a B-mode image of a femoral region on a display part of an information device.

FIG. 15C is a diagram illustrating an example in which a B-mode image on which nerve-emphasized data of a femoral region is superimposed is displayed on the display part after an independent display instruction.

FIG. 16A is a diagram illustrating a B-mode image including femoral nerves on a display part of an ultrasonic diagnostic apparatus.

FIG. 16B is a diagram illustrating a B-mode image including femoral nerves on the display part of the information device.

FIG. 16C is a diagram illustrating an example in which, on a display part of an ultrasonic diagnostic apparatus, a “B-mode image on which no nerve-enhancing data is superimposed” and a “B-mode image on which the nerve-enhancing data is superimposed” are displayed side by side after an independent display instruction.

FIG. 16D is a diagram illustrating an example in which a “B-mode image on which nerve-emphasized data is superimposed” is displayed on the display part after an independent display instruction.

FIG. 17 is a view illustrating an example in which a “B-mode image on which nerve enhancement data is not superimposed” is displayed on the display part of the ultrasonic diagnostic apparatus, and a message is superimposed and displayed after an independent display instruction.

### DETAILED DESCRIPTION OF THE EMBODIMENT

Hereinafter, one or more embodiments of the present invention will be described with reference to the drawings. However, the scope of the invention is not limited to the disclosed embodiments.

Hereinafter, an embodiment of the present invention will be described in detail with reference to the drawings.

### First Embodiment

The embodiment described below is directed to the case where, using the embodiment's ultrasonic image diagnostic system 1, a doctor on duty and a Cardiovascular Medicine specialist at a remote site evaluate the presence or absence of an aortic aneurysm as part of an ultrasonic examination for identifying the condition of a shock patient.

Specifically, the doctor on duty visually diagnoses that there is no aortic aneurysm based on the B-mode ultrasonic moving image captured by the operator. In response, the medical specialist using the information terminal 3 instructs the ultrasonic diagnostic apparatus 2 operated by the doctor on duty to display an independent ultrasonic image in order to obtain additional information necessary for accurate diagnosis. The ultrasonic diagnostic apparatus 2 generates a still image of a B-mode ultrasonic image and transfers it to the information terminal 3. The medical specialist measures the blood vessel diameter on the still image displayed on the display part 51 of the information terminal 3, and diagnoses an aortic aneurysm based on the measurement value of the blood vessel diameter including a thrombus portion. An independent display cancellation instruction is given to the ultrasonic diagnostic apparatus 2, and the doctor on duty is prompted to reevaluate the aortic diameter in a state where the same B-mode image as that of the ultrasonic diagnostic apparatus 2 is synchronously displayed on the display part 51.

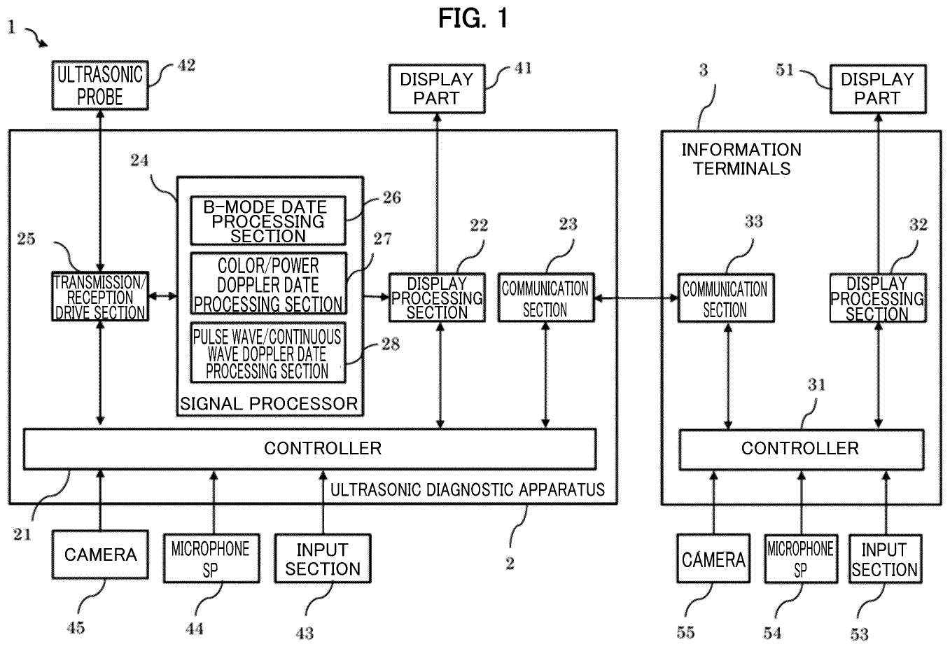

Here, the configuration of an ultrasonic image diagnostic system 1 according to an embodiment will be described with reference to FIG. 1. The ultrasonic image diagnostic system 1 includes an ultrasonic diagnostic apparatus 2 and an information terminal 3, which are connected to each other via a network such as a wireless/wired LAN, mobile communication, or the Internet. In the following description, the ultrasonic diagnostic apparatus 2 is a cart-type ultrasonic diagnostic apparatus, and the information terminal 3 is a PC including a display that supports touch operation on its screen.

The ultrasonic diagnostic apparatus 2 includes an ultrasonic probe 42, a transmission/reception driver 25, a signal processor 24, a controller 21, a display processor 22, a display part 41, a communication section 23, an input section 43, a camera 45, and a microphone SP44. The signal processing section 24 includes a B-mode data processing section 26, a color/power Doppler data processing section 27, and a pulse wave/continuous wave Doppler data processing section 28.

The ultrasonic probe 42 includes an ultrasonic probe, irradiates a subject with an ultrasonic beam by a drive signal of the transmission/reception drive section 25, receives a reflected ultrasonic wave (echo) from the subject, converts it into an ultrasonic signal, and supplies it to the transmission/reception drive section 25. The transmission/reception drive unit 25 supplies a drive signal to the ultrasonic probe 42 under the control of the controller 21, receives an ultrasonic signal of a reflected ultrasonic wave (echo) from the ultrasonic probe 42, and supplies the ultrasonic signal to the signal processing unit 24.

The signal processing unit 24 generates an ultrasonic image such as a B-mode image, a color Doppler image, or a pulse wave Doppler image based on the ultrasonic signal supplied from the transmission/reception driving unit 25. Further, the signal processing unit 24 performs enhancement processing of a puncture needle, a nerve, and a blood vessel region of the ultrasonic image. The display processing section 22 performs various kinds of image processing necessary for displaying the ultrasonic image generated by the signal processing section 24 on the display part 41 and processing related to an interface. The display part 41 displays the display image data including the ultrasonic image output from the display processing part 22. A touch screen (input section 43) is provided on a display surface of the display part 41 so that a tap operation by the user can be detected. Instead of the tap operation of the button by the user, the operation instruction of the user may be performed by a mouse click or the like of the button.

The communication section 23 is an input/output unit for wireless/wired LAN and mobile communication that communicates with the information terminal 3. The camera 45 captures an image of the user (doctors on duty) of the ultrasonic diagnostic apparatus 2 when the user (specialist) of the information terminal 3 has a video chat. Furthermore, the camera 45 can also take an image of a subject against which the ultrasonic probe is abutted and notify the information terminal 3 of a video that indicates the posture of the subject and the position and angle of the ultrasonic probe at the time of imaging. The microphone SP44 is a microphone and a speaker for a voice chat with a user (specialist) of the information terminal 3. The input section 43 is an input section for an operation instruction of a user (doctors on duty).

Based on an operation instruction input from the input section 43, the controller 21 controls driving of the ultrasonic probe 42, display of an ultrasonic image on the display part 41, and communication with the information terminal 3 via the communication section 23.

The information terminal 3 includes an input section 53, a camera 55, a microphone SP54, a controller 31, a display processing unit 32, a display part 51, and a communication section 33.

The communication section 33 is an input/output unit for wireless/wired LAN and mobile communication that communicates with the ultrasonic diagnostic apparatus 2. The display processing section 32 performs various kinds of image processing necessary for displaying, on the display part 51, an ultrasonic image notified from the ultrasonic diagnostic apparatus 2 via the communication section 33, and processing related to a user interface.

The display part 51 displays the display image data including the ultrasonic image output from the display processing part 32. A touch screen (input section 53) is provided on the display surface of the display part 51 so that a tap operation by the user can be detected. Instead of the tap operation of the button by the user, the user may perform an operation instruction by a mouse click or the like of the button.

The camera 55 captures an image of a user (medical specialist) of the information terminal 3 when performing a video chat with a user (doctors on duty) of the ultrasonic diagnostic apparatus 2. The microphone SP54 is a microphone and a speaker used for a voice chat with a user (doctors on duty) of the ultrasonic diagnostic apparatus 2. The input section 53 is an input section for a user (medical specialist) to give an operation instruction.

The controller 31 controls display of an ultrasonic image on the display part 51 and communication with the ultrasonic diagnostic apparatus 2 via the communication section 33, based on an operation instruction input from the input section 53.

More specifically, the ultrasonic diagnostic apparatus 2 and the information terminal 3 according to the embodiment are implemented by an information processing apparatus (computer) including a central processing unit (CPU), a read only memory (ROM), a random access memory (RAM), and a large-capacity recording medium such as a hard disk drive (HDD) or a solid state drive (SSD).

The CPU reads a program stored in the ROM or the HDD/SSD, develops the program in the RAM, and implements the function of the ultrasonic diagnostic apparatus 2 and the information terminal 3 in accordance with the developed program. The RAM forms a work area for temporarily storing various programs to be executed by the CPU and data related to these programs. The ultrasonic image diagnostic program may be stored in the form of a computer-readable program code and supplied by a recording medium, or may be transmitted and supplied via a network.

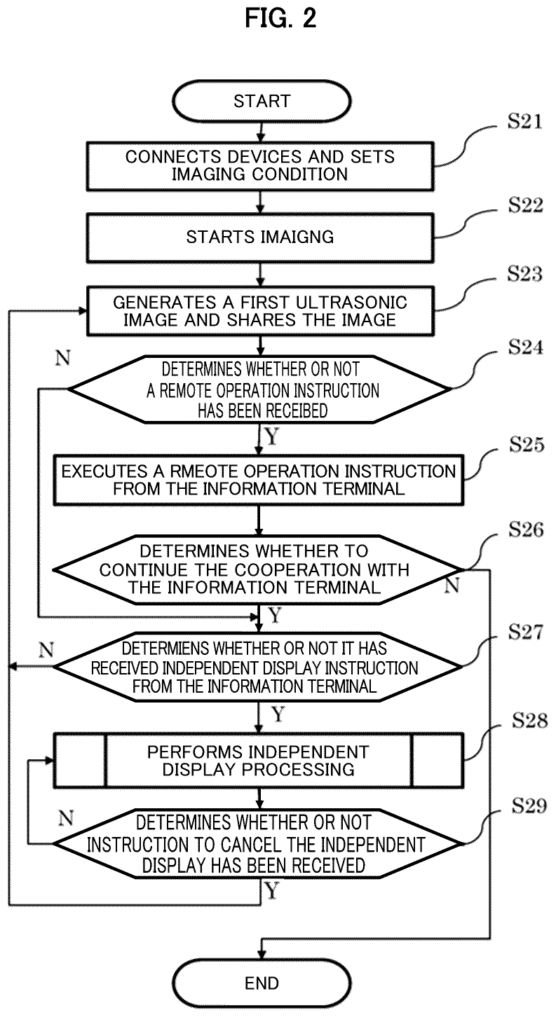

Next, the operation of the ultrasonic image diagnostic system 1 of the embodiment will be described with reference to the flowchart of FIG. 2. Note that in the present example, processing from step S24 to step S26 in which the ultrasonic diagnostic apparatus 2 is remotely operated from the information terminal 3 is not performed.

In step S21, the ultrasonic image diagnostic system 1 connects devices and sets imaging condition. Specifically, the doctor on duty connect the ultrasonic probe 42 and the camera 45 to the ultrasonic diagnostic apparatus 2 in the examination room and start the ultrasonic diagnostic apparatus 2. On the other hand, the medical specialist activates a notebook PC (information terminal 3) including a camera 55 (Web camera) and a touch-operable monitor (display part 51 and input section 53) from home, and remotely connects the notebook PC to the ultrasonic diagnostic apparatus 2.

Next, a request for permission for the next two cooperative functions is made from the information terminal 3 to the ultrasonic diagnostic apparatus 2, and the ultrasonic diagnostic apparatus 2 permits the request to the information terminal 3.

One is to share an ultrasonic image, a camera image, a mouse pointer, and drawing data by a drawing tool between the display part 41 of the ultrasonic diagnostic apparatus 2 and the display part 51 of the information terminal 3. Sharing of an ultrasonic image is performed by live transmission of the ultrasonic image from the ultrasonic diagnostic apparatus 2 to the information terminal 3. When a camera image, a mouse pointer, or a draw tool on the other apparatus is used, the drawing data is transmitted live to the other apparatus for sharing. The other is to transmit a remote operation command from the information terminal 3 to the ultrasonic diagnostic apparatus 2.

Furthermore, a web meeting tool is separately activated, and voice or chat communication is also made executable. Further, the doctor on duty registers information on the conveyed patient in the shock state in the patient database of the ultrasonic diagnostic apparatus 2, and selects the B mode as a condition of the imaging mode.

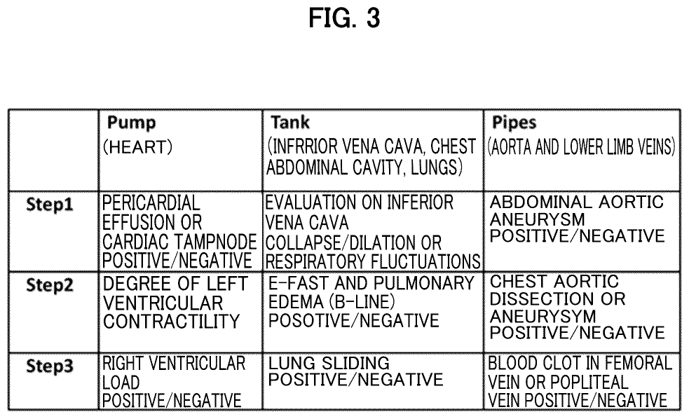

In step S22, the ultrasonic diagnostic apparatus 2 starts imaging of an ultrasonic image. In detail, the doctor on duty start RUSH (Rapid Ultrasonic in SHock) exam as the ultrasonic examination. As illustrated in FIG. 3, the inspection content is to evaluate each item of Pump (heart), Tank (lungs, inferior vena cava, chest, and abdominal cavity), and Pipes (aorta and lower limb veins) in three steps. The doctor on duty diagnoses the pathological condition of shock (cardiogenic/occlusive/hypovolemic/distributive) by this inspection.

In step S23, the ultrasonic diagnostic apparatus 2 generates a first ultrasonic image and shares the ultrasonic image with the information terminal 3.

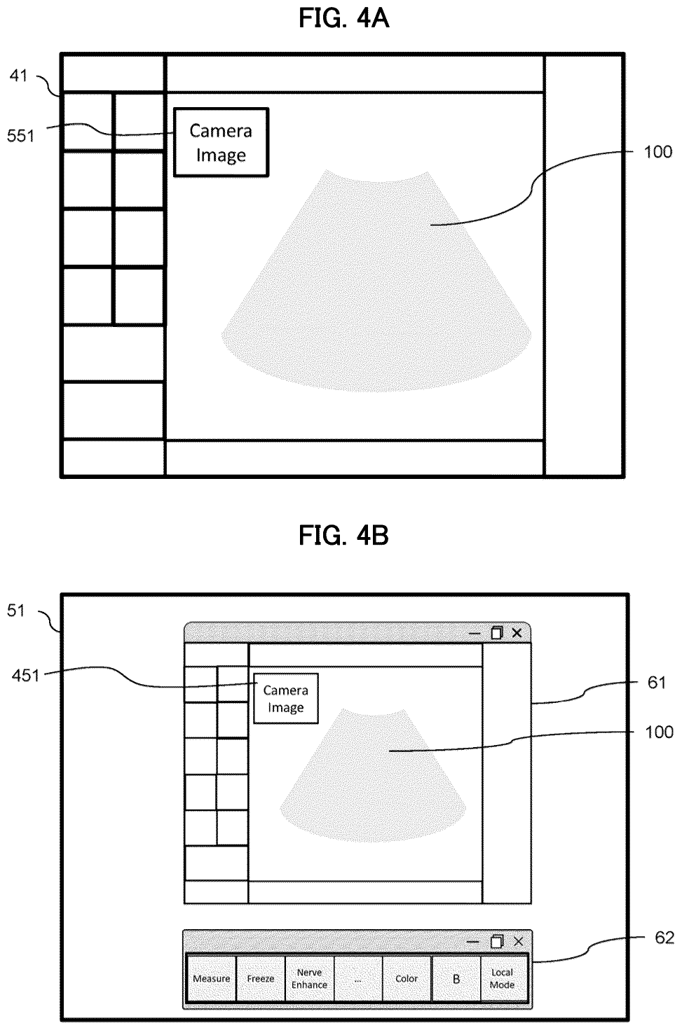

Specifically, the ultrasonic diagnostic apparatus 2 scans the body surface of the patient using the connected convex probe (ultrasonic probe 42), generates a first ultrasonic image in B-mode based on the acquired ultrasonic signals, and displays the image as an ultrasonic image 100 as illustrated in FIG. 4A on the display part 41.

Furthermore, the controller 21 of the ultrasonic diagnostic apparatus 2 transmits the first ultrasonic image from the communication section 23 to the communication section 33 of the information terminal 3. In the information terminal 3, the controller 31 sends the received first ultrasonic image to the display processor 32, and as illustrated in FIG. 4B, performs conversion processing for display and displays the image as the ultrasonic image 100 on the observation image 61 of the display part 51.

Here, display screens of the ultrasonic diagnostic apparatus 2 and the information terminal 3 will be described with reference to FIGS. 4A and 4B. FIG. 4A is a diagram illustrating display content of the display part 41 of the ultrasonic diagnostic apparatus 2. The display part 41 displays the ultrasonic image 100, and at the time of video chat, displays a captured image 551 of the medical specialist captured by the camera 55 of the information terminal 3.

FIG. 4B is a view illustrating display content of the display part 51 of the information terminal 3. The display part 51 displays an observation image 61, which is the shared display content of the display part 41 of the ultrasonic diagnostic apparatus 2, and a remote operation floating window 62 for remotely instructing the ultrasonic diagnostic apparatus 2 from the information terminal 3. As the observation image 61, the shared ultrasonic image 100 is displayed, and at the time of video chat, a captured image 451 of the doctor on duty or the subject (with which the ultrasonic probe has been brought into contact) captured by the camera 45 of the ultrasonic diagnostic apparatus 2 is displayed.

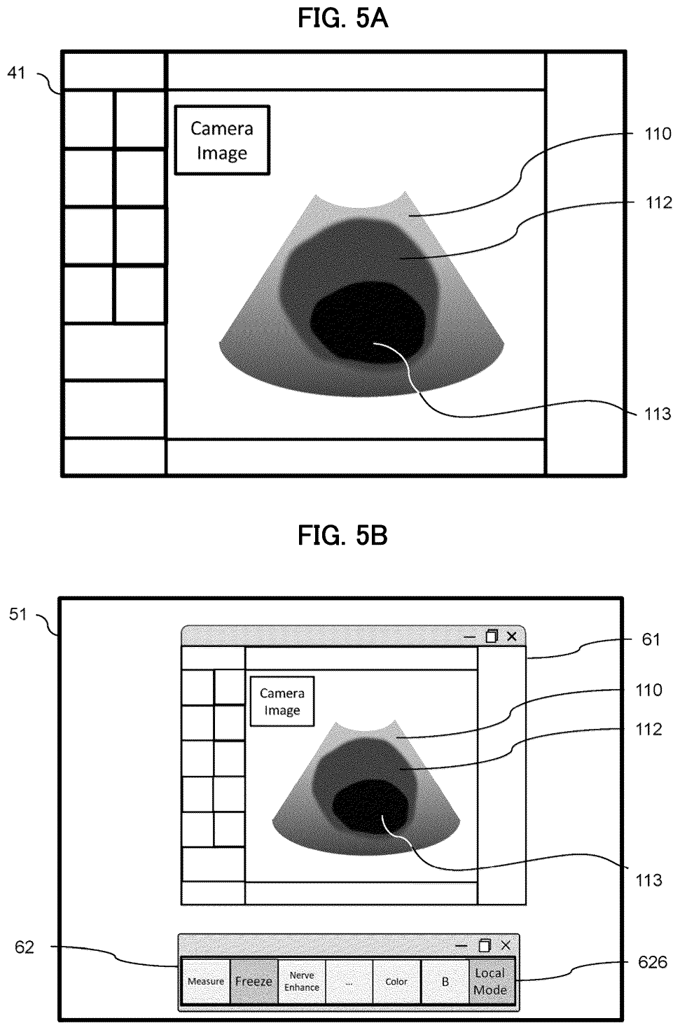

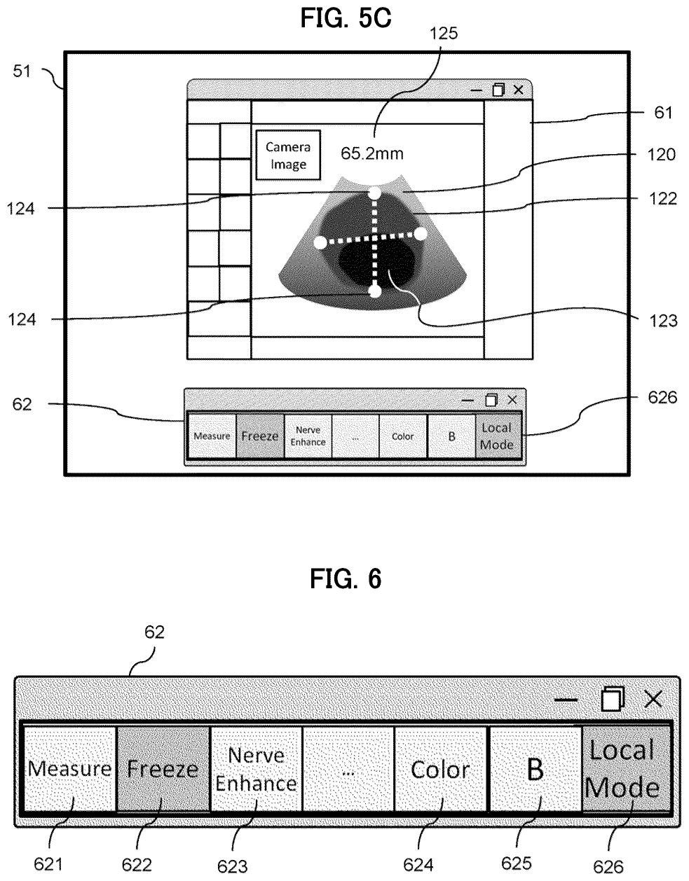

FIGS. 5A to 5C are diagrams illustrating ultrasonic images of the abdominal Step1 when the doctor on duty performs an inspection (evaluation of the abdominal aortic diameter) of the aorta related to Pipes (blood vessels) and diagnoses the presence or absence of an abdominal aortic aneurysm in the inspection content illustrated in FIG. 3.

FIG. 5A is a diagram illustrating display content of the display part 41 of the ultrasonic diagnostic apparatus 2. The display part 41 displays an ultrasonic image 110 (B-mode image) of the aorta. A thrombus region 112 and a blood flow region 113 are illustrated in the ultrasonic image 110. The doctor on duty determines that the state is normal (the vascular caliber is smaller than 5 cm) based on the blood flow region 113 in the display part 41.

FIGS. 5B to 5C are diagrams illustrating display content of the display part 51 of the information terminal 3. As will be described later, FIG. 5B illustrates a state in which an ultrasonic image 110 (B-mode image) of the main artery is displayed in synchronization with FIG. 5A. FIG. 5C illustrates a state in which a blood vessel diameter is measured after the screen is frozen by giving an instruction to operate the remote operation floating window 62.

Returning to FIG. 2, in step S24, the ultrasonic diagnostic apparatus 2 determines whether or not a remote operation instruction (remote operation command) has been received from the information terminal 3. If it has been received (Y in step S24), the process proceeds to step S25. If not received (N in step S24), the process proceeds to step S27.

The remote operation instruction from the information terminal 3 refers to a remote operation command to be transmitted from the information terminal 3 to the ultrasonic diagnostic apparatus 2. Specifically, the remote operation command is notified from the information terminal 3 to the ultrasonic diagnostic apparatus 2 in response to a user operation on the remote operation floating window 62 of the display part 51 of the information terminal 3.

FIG. 6 is a diagram illustrating details of the remote control floating window 62. In the remote control floating window 62, for example, commands to instruct start/stop of imaging modes such as a B mode, a color Doppler mode, and a pulse wave Doppler mode, and commands to specify a depth or focus position, display luminance, and a frame rate are arranged. In addition, an operation button of a command for instructing highlighting of a puncture needle, a nerve, and a blood vessel region and application/cancellation of various kinds of filter processing is arranged.

In FIG. 6, a “Measure” button 621, a “Freeze” button 622, a “Nerve Enhance” button 623, a “Color” button 624, a “B” button 625, and a “Local Mode” button 626 are provided. The “Measure” button 621 instructs start/end of a measurement mode for measuring an actual distance between two points on an ultrasonic image. The “Freeze” button 622 instructs start/end of the still image display mode of the observation image 61 of the information terminal 3.

The “Nerve Enhance” button 623 gives an instruction to start/end the neural enhancement mode. The “Color” button 624 instructs the start/end of color Doppler mode imaging. The “B” button 625 gives an instruction to start/end B-mode imaging. The “Local Mode” button 626 instructs start/end of processing for generating and transmitting a second ultrasonic image used for independent display in the information terminal 3.

When the input section 53 detects the user's tap on the remote operation floating window 62, the controller 31 of the information terminal 3 notifies the ultrasonic diagnostic apparatus 2 of a remote operation instruction (remote operation command) corresponding to the operated button.

Returning to FIG. 2, in step S25, the ultrasonic diagnostic apparatus 2 executes a remote operation instruction (remote operation command) from the information terminal 3.

In step S26, the ultrasonic diagnostic apparatus 2 determines whether to continue the cooperation with the information terminal 3. That is, the ultrasonic diagnostic apparatus 2 confirms the connection of the information terminal 3. In a case where it is determined that the information terminal 3 is in the unconnected state, the ultrasonic diagnostic apparatus 2 determines not to continue cooperation (N in step S26), and the process of FIG. 2 ends. When it is determined that the information terminal 3 is in the connected state, the ultrasonic diagnostic apparatus 2 determines to continue cooperation (step S26), and the process proceeds to step S27.

In step S27, the ultrasonic diagnostic apparatus 2 determines whether or not it has received an independent display instruction from the information terminal 3, which has been notified in response to an operation instruction of the “Local Mode” button 626 by the user (medical specialist) of the information terminal 3. If the independent display instruction has been received (Y in step S27), the process proceeds to step S28, and if the independent display instruction has not been received (N in step S27), the process returns to step S23.

For example, the medical specialist notices the presence of a thrombotic region 122 in the ultrasonic image of the abdominal aorta indicated by the observation image 61 in the FIG. 5B, and taps the “Local Mode” button 626 and the “Freeze” button 622 in the remote operation floating window 62. Thus, the information terminal 3 notifies the ultrasonic diagnostic apparatus 2 of an independent display instruction (an instruction to generate and transmit a still image).

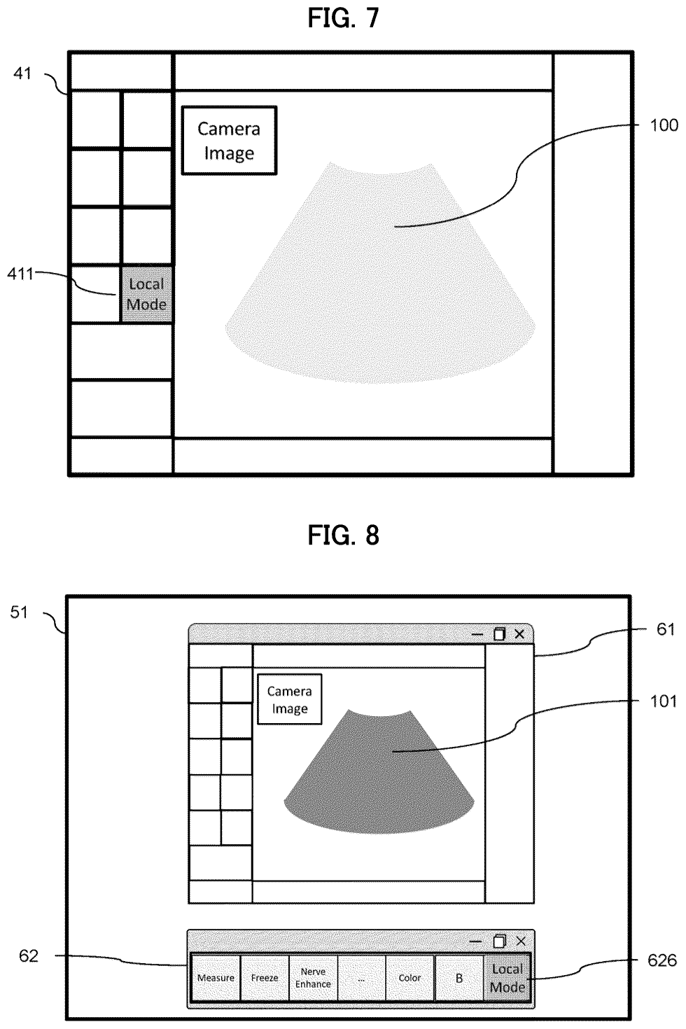

It has been described above that the “Local Mode” button 626 for setting the independent display instruction function and the independent display cancellation instruction function is provided in the remote operation floating window 62 of the information terminal 3. However, as shown in FIG. 7, the display part 41 of the ultrasonic diagnostic apparatus 2 may be provided with a “Local Mode” button 411 for setting the independent display instruction function and the independent display cancellation instruction function of the screen. That is, the ultrasonic diagnostic apparatus 2 is provided with an operation button that suppresses a change in the display function of the display part 41 from the information terminal 3. Thus, it is possible to prevent a treatment error of the user (doctors on duty) of the ultrasonic diagnostic apparatus 2 due to a change in the display content.

A “Local Mode” button 626 in the remote operation floating window 62 in FIG. 5B and a “Local Mode” button 411 in FIG. 7 are operation buttons that perform a toggle operation by a tap of the user, the toggle operation serving as both an independent display instruction function and an independent display cancellation instruction function. However, the operation instruction method is not limited thereto.

As another operation instruction method, for example, an interface for an independent display instruction and an interface for an independent display cancellation instruction may be separately provided, and a desired operation button in the remote operation floating window 62 may be tapped in a state where the independent display instruction or the independent display cancellation instruction is performed by tapping these interfaces.

Alternatively, in the remote operation floating window 62, when the button is tapped for the first time, the operation may be executed as an operation in a state where the independent display is instructed, and when the button is tapped again (for the second time), the operation may be executed as an operation in a state where the independent display release is instructed. In this case, it is not necessary to provide a user interface for the independent display instruction/cancellation instruction, such as the “Local Mode” button 626.

Such a toggle operation of the operation button means that each time the button is tapped, a transition is made between the states of the independent display instruction and the independent display cancellation instruction. It is desirable that the operation instruction button is displayed on the operation screen in a display mode (color, brightness, opacity, image, note, icon, or the like) indicating the state of the independent display instruction or the independent display cancellation instruction. Alternatively, a predetermined operation (double tap, long press, or the like) on each operation button may be assigned as the independent display instruction function or the independent display cancellation instruction function.

When a desired operation (other than the independent display instruction/cancellation instruction) of the remote operation floating window 62 is executed in a state where the independent display instruction is executed, the operation is executed as an operation instruction for displaying the second ultrasonic image only on the information terminal 3. When the independent display cancellation instruction is executed, the first ultrasonic image generated by the ultrasonic diagnostic apparatus 2 is displayed on the display part 51 of the information terminal 3, and the synchronous display (cooperation) is recovered.

Furthermore, in a case where a desired operation of the remote-control floating window 62 is executed in a state where the independent display cancellation instruction has been executed, the following operation is performed. That is, it is executed as a remote operation to the ultrasonic diagnostic apparatus 2, and the first ultrasonic image generated by the remote operation is displayed on the display part 41 of the ultrasonic diagnostic apparatus 2 and the display part 51 of the information terminal 3.

Referring back to FIG. 2, in step S28, the ultrasonic diagnostic apparatus 2 performs independent display processing, which will be described in detail later. That is, the ultrasonic diagnostic apparatus 2 generates an ultrasonic image for independent display (second ultrasonic image) and transmits the ultrasonic image to the information terminal 3. For example, the still image 120 is displayed on the observation image 61 of the display part 51 of the information terminal 3 as the second ultrasonic image.

Thus, the information terminal 3 independently displays the second ultrasonic image without changing the first ultrasonic image displayed by the ultrasonic diagnostic apparatus 2 by the operation of the “Local Mode” button 626 as the independent display instruction unit.

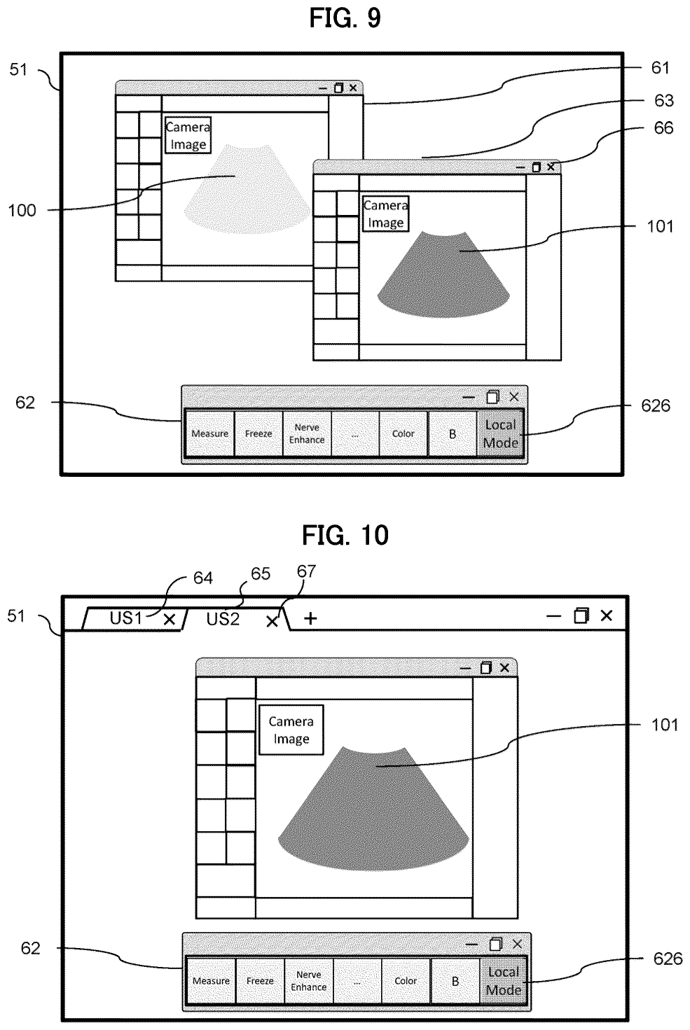

As variations of the independent display on the display part 51 of the information terminal 3, any of the following displays may be provided.
As illustrated in FIG. 8, the ultrasonic image 101 for independent display, which is the second ultrasonic image, is displayed so as to overwrite the area of the ultrasonic image 100 that has been synchronously displayed.
   That is, synchronous display (cooperative operation) with the ultrasonic diagnostic apparatus 2 is interrupted, and the second ultrasonic image is displayed.
As illustrated in FIG. 9, an ultrasonic image 101 for independent display, which is a second ultrasonic image, is displayed on a window (observation image 63) different from the window (observation image 61) on which synchronous display is performed.
As shown in FIG. 10, an ultrasonic image 101 for independent display, which is a second ultrasonic image, is displayed in a tab 65 different from the tab 64 of the window that is synchronously displayed.

Referring back to FIG. 2, in step S29, the ultrasonic diagnostic apparatus 2 determines whether or not an instruction to cancel independent display has been received from the information terminal 3. If not received (N in step S29), the process returns to step S28. If the synchronization display has been received (Y in step S29), the ultrasonic diagnostic apparatus 2 recovers the synchronization display, and then the process returns to step S23.

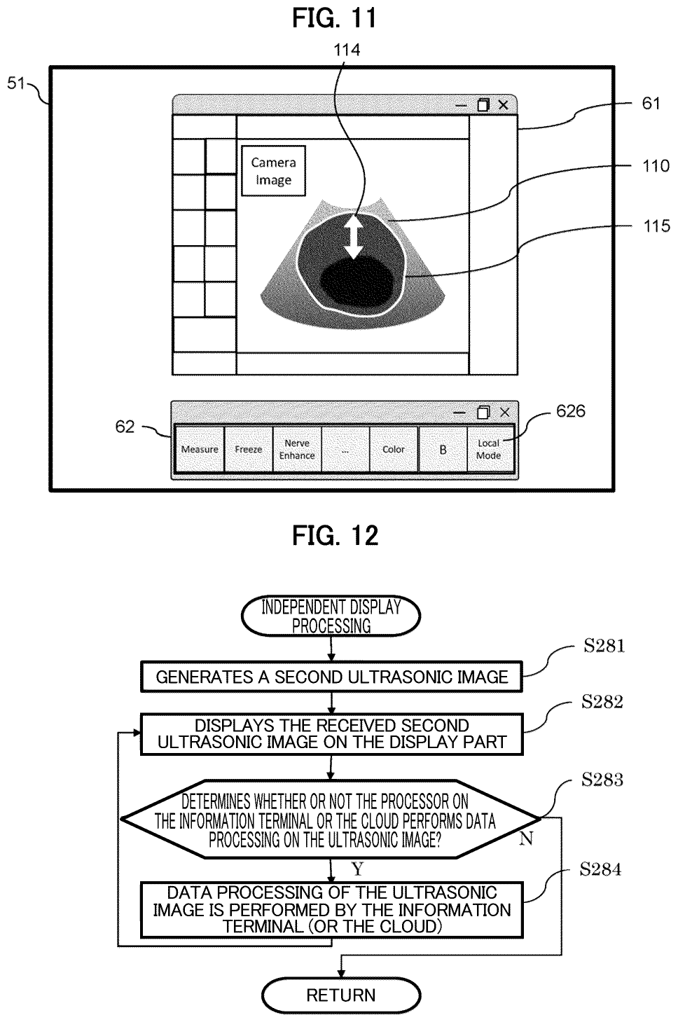

When the medical specialist taps the independent display release button (the “Local Mode” button 626) on the display part 51 of the information terminal 3, the information terminal 3 issues an independent display cancellation instruction to the ultrasonic diagnostic apparatus 2. As shown in FIG. 11, the first ultrasonic image 110 after the synchronous display is restored is displayed on the information terminal 3, and the medical specialist put marks 115 on the thrombus region and a part of the boundary thereof on the first ultrasonic image 110 using the draw tool. Here, the draw tool is a function of drawing a curve or a figure by designating a coordinate series on a screen using a pointing device or an operator's finger.

The ultrasonic diagnostic apparatus 2 displays the marked ultrasonic image on the display part 41 of the ultrasonic diagnostic apparatus 2. That is, the specialist and the doctor on duty can share the marked ultrasonic image. Thus, the specialist informs the doctor on duty that the aortic diameter is underestimated, prompting re-evaluation. The doctor on duty re-evaluates the aortic diameter and diagnoses abdominal aortic aneurysm.

As an independent display cancellation instruction method, independent display cancellation can be instructed by any of the following sequences in accordance with an implementation method of independent display.
In FIG. 8, the ultrasonic image 100 to be synchronously displayed is displayed so as to overwrite the area of the ultrasonic image 101 for independent display.
In FIG. 9, the user interface (66) for closing the window (observation image 63) displaying the ultrasonic image 101 for independent display, which is the second ultrasonic image, is tapped to cancel the independent display.
In FIG. 10, the user interface (67) for closing the tab 65 for displaying the ultrasonic image 101 for independent display, which is the second ultrasonic image, is tapped to cancel the independent display.

Next, details of the independent display processing in step S28 of FIG. 2 will be described with reference to the flowchart of FIG. 12.

In step S281, the ultrasonic diagnostic apparatus 2 generates a second ultrasonic image and transmits the second ultrasonic image to the information terminal 3.

In step S282, the information terminal 3 displays the received second ultrasonic image on the display part 51.

In step S283, the information terminal 3 determines whether or not the processor on the information terminal 3 (or the cloud) performs data processing of the ultrasonic image such as measurement on the second ultrasonic image. When the data processing of the ultrasonic image is performed (step S283), the processing proceeds to step S284. If the ultrasonic image is not to be processed (N in step S283), the process in FIG. 12 is terminated, and the process returns to step S29 in FIG. 2.

In step S284, data processing of an ultrasonic image is performed by the information terminal 3 (or a processor on a cloud). Next, the process returns to step S282, and the information terminal 3 displays the data-processed ultrasonic image on the display part 51.

Hereinafter, the process of FIG. 12 will be specifically described with reference to an example of a process of measuring the aortic diameter including a thrombotic region on the still image in FIG. 5C.

The measurement processing between the two specified points performed as the independent display processing by the information terminal 3 in step S284 is executed by the processor on the information terminal 3 (or the cloud) by specifying the measurement position 124 on the observation image 61 with the pointing device or the fingertip.

At this time, it is assumed that the data regarding the pixel size is acquired in advance when the first ultrasonic image is acquired from the ultrasonic diagnostic apparatus 2 (as a part of data for displaying the first ultrasonic image).

In step S282, the information terminal 3 displays the aortic diameter of 65.2 mm as the measurement value 125 obtained in step S284. Since the measured aortic diameter is larger than 50 mm, a specialist diagnoses it as an aortic aneurysm. At this time, FIG. 5A is displayed on the ultrasonic diagnostic apparatus 2.

In step S283, since the information terminal 3 has already completed the measurement processing and there is no other processing to be executed by the processor on the information terminal 3 (or the cloud), the information terminal 3 ends the independent display processing and returns to step S29 in FIG. 2.

Next, a sequence for sharing the drawing data by the draw tool illustrated in FIG. 11 between the ultrasonic diagnostic apparatus 2 and the information terminal 3 will be described in detail. That is, on the display part 41 of the ultrasonic diagnostic apparatus 2 (or the display part 51 of the information terminal 3), the coordinate series is designated by the pointing device or the fingertip of the operator. Thus, a free curve or a figure is drawn on the display part 41 of the ultrasonic diagnostic apparatus 2 (or the display part 51 of the information terminal 3), and drawing data is shared.

At this time, the coordinate series is shared between the display part 41 of the ultrasonic diagnostic apparatus 2 and the display part 51 of the information terminal 3. That is, the coordinate series data drawn on the display part 41 of the ultrasonic diagnostic apparatus 2 (or the display part 51 of the information terminal 3) by using the draw tool is transferred from the ultrasonic diagnostic apparatus 2 to the information terminal 3 (or from the information terminal 3 to the ultrasonic diagnostic apparatus 2).

Then, it is drawn on the ultrasonic image 110 on the display part 51 of the information terminal 3 (or the display part 41 of the ultrasonic diagnostic apparatus 2). The display position of the coordinate series on the ultrasonic image 110 on the display part 41 of the ultrasonic diagnostic apparatus 2 is the same as the display position on the ultrasonic image 110 on the display part 51 of the information terminal 3.

The coordinate series data drawn on the display part of the device (the information terminal 3 or the ultrasonic diagnostic apparatus 2) that the user himself/herself is directly operating can be made non-display by the user himself/herself performing a deleting operation. The deletion operation may be performed by designating the type and range of a coordinate series to be deleted using a pointing device or a finger. In addition, in the deletion operation, a type of a coordinate series that is a target may be selected by the user, and then the coordinate series may be deleted by executing a deletion instruction via a context menu or a deletion button.

The deletion may be automatically performed when a preset time has elapsed after the drawing, without requiring an explicit deletion operation by the user. Note that a plurality of free curves or graphics drawn by the information terminal 3 or the ultrasonic diagnostic apparatus 2 may be displayed in different display modes so as to be easily identified, or a curve or a graphic drawn by the ultrasonic diagnostic apparatus 2 and a curve or a graphic drawn by the information terminal 3 may be displayed in different display modes.

For example, as the display mode, at least one of the color, the line type, the thickness, the brightness, the opacity, and the style (none/arrow/circle, etc) of the end portion may be displayed so as to be different from each other. Alternatively, an annotation indicating the creating device or the creator may be displayed in the vicinity of each drawn free curve or graphic. In addition, a setting menu related to a drawing (coordinate series designation) function of a curve or a figure using a draw tool may be prepared, and for example, a default value of a display mode, a drawing period, sharing/non-sharing of drawing data, or the like may be set.

### Second Embodiment

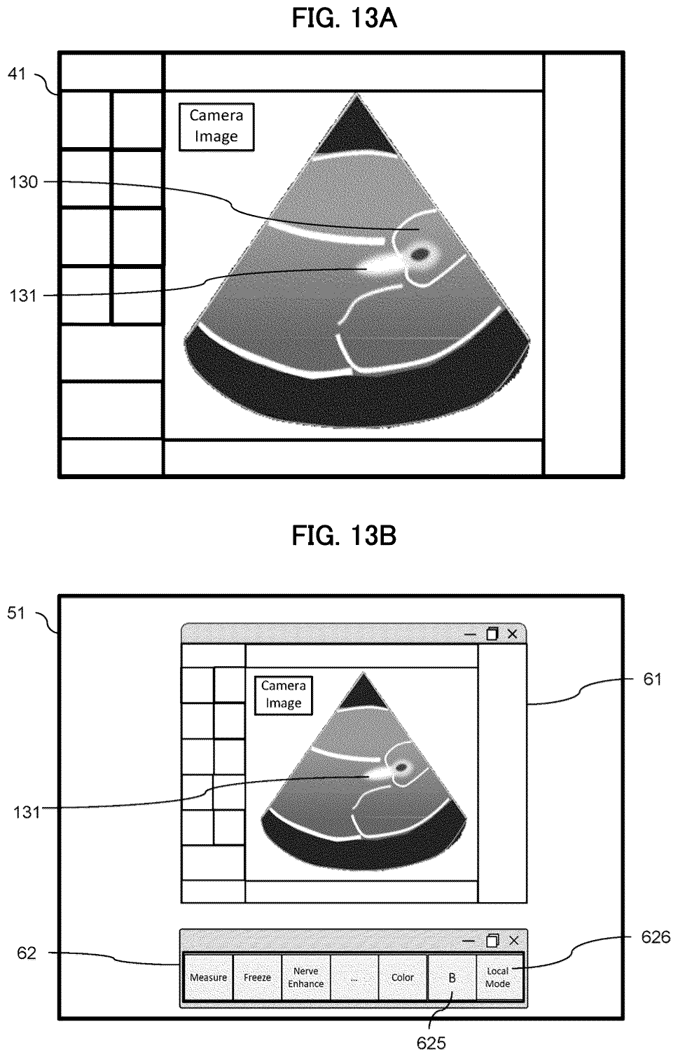

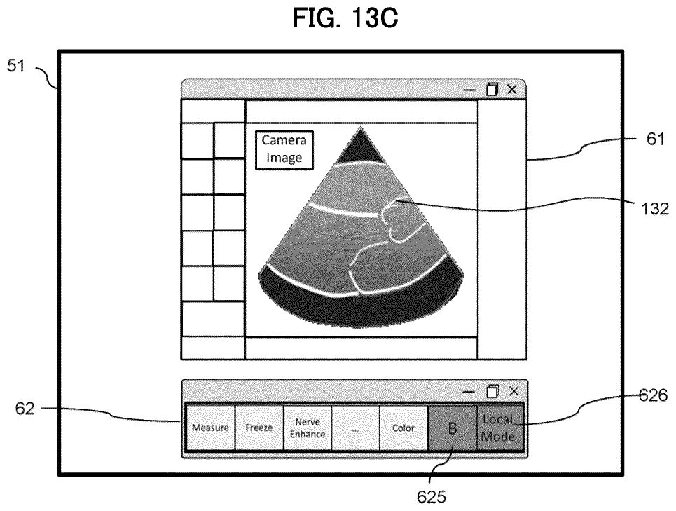

Next, with reference to FIGS. 13A to 13C, a description will be given of a case where, using the ultrasonic image diagnostic system 1, a doctor on duty and a Cardiovascular Medicine specialist at a remote site evaluate the presence or absence of aortic dissection in an ultrasonic image as part of an ultrasonic examination for identifying the condition of a shock patient.

Specifically, the doctor on duty evaluates the presence or absence of aortic valve regurgitation in the color Doppler image (to be precise, an image obtained by superimposing color Doppler data on a B-mode image) illustrated in the FIG. 13A, which is captured by the operator using the ultrasonic diagnostic apparatus 2. At this time, the specialist in Cardiovascular Medicine using the information terminal 3 gives an independent display instruction to the ultrasonic diagnostic apparatus 2 in order to evaluate the presence or absence of the flap 132 in the aortic region 130. The ultrasonic diagnostic apparatus 2 generates a B-mode image (on which a color Doppler image is not superimposed), transfers the B-mode image to the information terminal 3, and displays the B-mode image on the display part 51 (FIG. 13C). The medical specialist searches for the flap 132 in the aortic region 130 on the B-mode image in FIG. 13C displayed on the display part 51 of the information terminal 3.

Hereinafter, detailed description will be given with reference to the flowchart of FIG. 2.

In step S21 and step S22, the ultrasonic diagnostic imaging system 1 designates the color Doppler mode as the imaging mode, and starts imaging. A B-mode image and a color Doppler image are captured live in a color Doppler mode, and the color Doppler image is displayed live in a state of being superimposed on the B-mode image.

In step S23, the ultrasonic image diagnostic system 1 generates a color Doppler image as a first ultrasonic image, and as illustrated in FIGS. 13A and 13B, displays the image on the display part 41 of the ultrasonic diagnostic apparatus 2 and the display part 51 of the information terminal 3, respectively, to share the ultrasonic image.

Specifically, the color Doppler image (first ultrasonic image) exemplified in the present embodiment is an image captured in a long-axis cross section of the left margin of the sternum using a sector probe. Then, a state in which color Doppler data indicating aortic valve regurgitation 131 is displayed in the aorta region 130 is shared live.

In order to evaluate the presence or absence of a thoracic aortic dissection/aneurysm in the Step2 of the inspection for Pipes (aorta and lower limb veins) in FIG. 3, the doctor on duty cause the color Doppler image in FIG. 13A to be displayed and checks whether the aortic valve regurgitation 131 has occurred. Although it is relatively easy to observe aortic valve regurgitation 131, it is an indirect finding of aortic dissection and requires a more direct finding to diagnose aortic dissection.

In step S24, since a remote operation from the information terminal 3 is not performed in the present example, the processes of step S25 and step S26 are not performed, and the process proceeds to step S27.

The specialist taps the “Local Mode” button 626 and the “B” button 625 in the remote control floating window 62 in order to observe the flap 132 in the aortic region 130, which is a definitive finding indicating aortic dissection. Here, the “Local Mode” button 626 is an operation button for executing an instruction related to independent display. The “B” button 625 is an operation button for giving an instruction to start/end ultrasonic imaging in the B mode.

Accordingly, in step S27, the ultrasonic image diagnostic system 1 determines that the independent display instruction has been received, and the process proceeds to step S28.

In step S28, the information terminal 3 instructs the ultrasonic diagnostic apparatus 2 to generate a B-mode image, and the ultrasonic diagnostic apparatus 2 transmits, to the information terminal 3, the B-mode image on which no color Doppler image is superimposed as the second ultrasonic image. Next, the information terminal 3 displays the transmitted B-mode image on the display part 51 as illustrated in FIG. 13C.

The specialist carefully searches for the flap 132 in the aortic region 130 on the B-mode image of the information terminal 3 illustrated in FIG. 13C, and finds it as a direct finding indicating aortic dissection. When finishing the search in the aortic region 130, the specialist taps the “Local Mode” button 626.

In step S29, in response to the reception of the independent display cancellation instruction from the information terminal 3, the ultrasonic diagnostic apparatus 2 returns to step S23, and returns the display on the display part 51 of the information terminal 3 to the state of the synchronous display of the color Doppler image in FIG. 13B. Next, the medical specialist informs the doctor on duty that the flap has been confirmed on the B-mode image, and consults about a treatment policy for aortic dissection together with the finding of aortic valve regurgitation confirmed on the color Doppler image.

### Third Embodiment

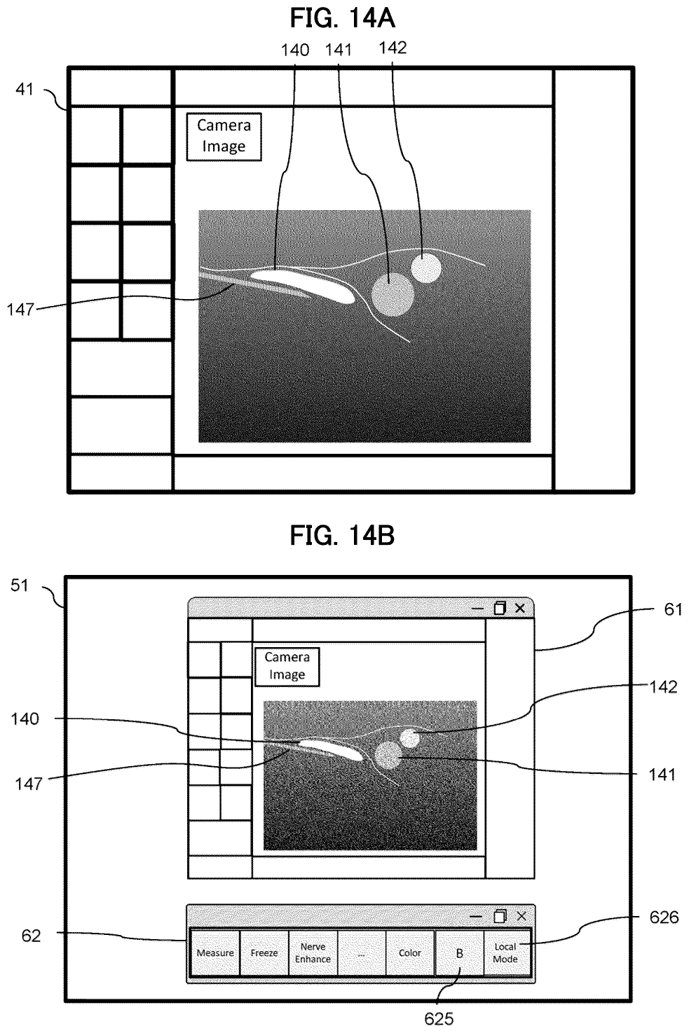

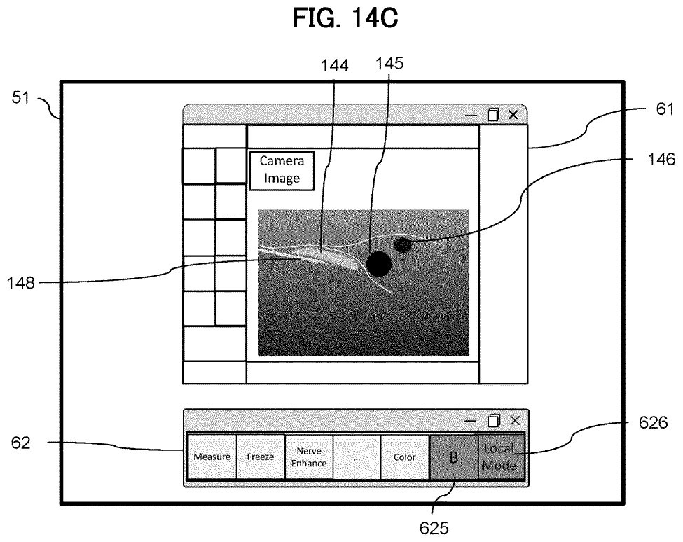

Next, an example of performing femoral nerve block on a femur fracture patient using the ultrasonic image diagnostic system 1 of the above-described embodiment will be described with reference to FIGS. 14A, 14B, and 14C. An ultrasonic diagnostic apparatus operated by an emergency doctor displays a B-mode image on which emphasis data related to a puncture needle or a nerve/blood vessel is superimposed, and an information terminal operated by an anesthesiologist at a remote place instructs independent display to display a B-mode image on which emphasis data is not superimposed.

Specifically, the emergency physician performs nerve block using the B-mode ultrasonic image on which the superimpose data of the puncture needle of the ultrasonic diagnostic apparatus 2, the superimpose data of the puncture target (femoral nerve), and the superimpose data of the tissue (blood vessel) that should not be punctured are superimposed. Thus, the emergency physician reliably performs femoral nerve block on the patient with a fractured femur.

The anesthesiologist using the information terminal 3 issues an independent display instruction to the ultrasonic diagnostic apparatus 2 to generate a normal B-mode image in order to evaluate whether the drug solution is accurately injected around the nerve. Here, the normal B-mode image means a B-mode image in which any of the superimposed displays of the puncture needle emphasis data, the nerve emphasis data, and the blood vessel emphasis data is disabled.

The ultrasonic diagnostic apparatus 2 transfers only the B-mode image on which the puncture needle emphasis/nerve emphasis/blood vessel emphasis data is not superimposed to the information terminal 3. The specialist advises about nerve block while checking the injection status of the liquid medicine on the B-mode image transferred from the ultrasonic diagnostic apparatus 2 and displayed on the display part 51 of the information terminal 3.

The following operation is performed in the ultrasonic image diagnostic system 1 of the present example. That is, it is assumed that the permission request regarding the linkage function between the information terminal 3 and the ultrasonic diagnostic apparatus 2 as shown in the following (I) to (iii) is performed from the information terminal 3 to the ultrasonic diagnostic apparatus 2, and the ultrasonic diagnostic apparatus 2 permits the information terminal 3.
(i) A function of sharing an ultrasonic image and a camera image between the display part 41 of the ultrasonic diagnostic apparatus 2 and the display part 51 of the information terminal 3 by transmitting the ultrasonic image live from the ultrasonic diagnostic apparatus 2 to the information terminal 3 or transmitting the camera images of the ultrasonic diagnostic apparatus 2 and the information terminal 3 live from each other.
(ii) A function of transmitting a remote operation command from the information terminal 3 to the ultrasonic diagnostic apparatus 2.
(iii) A function of performing voice communication via the microphone connected to the ultrasonic diagnostic apparatus 2 and the information terminal 3.

In addition, the emergency doctor registers information on the conveyed fracture patient in the patient database of the ultrasonic diagnostic apparatus 2, and selects the B mode as the imaging mode.

Hereinafter, the detailed description will be given with reference to the flowchart of FIG. 2.

In step S21, the emergency physician activates the ultrasonic diagnostic apparatus 2 with the ultrasonic probe 42 and the camera 45 connected to each other. On the other hand, a specialist activates a PC (information terminal 3) including the camera 55 and a touch-operable monitor (input section 53+display part 51) from a remote hospital, and remotely connects the PC to the ultrasonic diagnostic apparatus 2.

In step S22, an emergency doctor registers information on a patient in a DB, and then selects a preset and an imaging mode (B mode), and the ultrasonic diagnostic apparatus 2 starts imaging of an ultrasonic image. In addition, the emergency physician performs a prescan of the vicinity of the surgical field in a state in which each function of the puncture needle, the nerve, and the blood vessel enhancement is enabled.

The emergency physician starts puncturing while adjusting the positions of the ultrasonic probe and the puncture needle. In step S23, the ultrasonic diagnostic apparatus 2 generates a B-mode image as a first ultrasonic image on the basis of an ultrasonic signal acquired by scanning the body surface of the patient using the connected linear probe, and displays the B-mode image on the display part 41 of the ultrasonic diagnostic apparatus 2 as shown in FIG. 14A.

As illustrated in FIG. 14A, on the B-mode image, emphasis information 147 on the puncture needles, neural emphasis information 140 on the femoral nerves, arterial emphasis information 141 on the femoral arteries, and venous emphasis information 142 on the femoral veins are superimposed and displayed.

The emergency physician can advance the needle toward the femoral nerve, which is a target, while checking the position of the puncture needle and taking care not to puncture the femoral artery and vein. In addition, the emergency physician injects a drug at the time point when the puncture needle reaches the affected part.

In step S24, since a remote operation from the information terminal 3 is not performed in the present example, the processes of step S25 and step S26 are not performed, and the process proceeds to step S27.

The medical specialist taps the “Local Mode” button 626 and the “B” button 625 in the remote operation floating window 62. The “Local Mode” button 626 is an operation button for setting the independent display instruction function. The “B” button 625 is an operation button for giving an instruction to start/end ultrasonic imaging in the B mode. In order to disable the functions of the puncture needle emphasis, the nerve emphasis, and the blood vessel emphasis, the medical specialist taps only the “B” button 625 without tapping the instruction buttons related to various kinds of emphasis display.

Accordingly, in step S27, the ultrasonic image diagnostic system 1 determines that the independent display instruction has been received, and the process proceeds to step S28.

In step S28, the information terminal 3 instructs the ultrasonic diagnostic apparatus 2 to generate a B-mode image, and the ultrasonic diagnostic apparatus 2 transmits, to the information terminal 3, the B-mode image on which the puncture needle emphasis data, the nerve emphasis data, and the blood vessel emphasis data are not superimposed as the second ultrasonic image. Next, the information terminal 3 displays the transmitted B-mode image on the display part 51 as illustrated in FIG. 14C.

The medical specialist checks, on the B-mode image in FIG. 14C, whether the hypoechoic area indicating the liquid medicine is sufficiently spread around the femoral nerve 144. When confirming the spread of the liquid medicine, the medical specialist taps the “Local Mode” button 626.

In step S29, in response to reception of the independent display cancellation instruction from the information terminal 3, the ultrasonic diagnostic apparatus 2 returns to step S23. Then, the display of the display part 51 of the information terminal 3 is returned to the synchronous display state of the B-mode image (first ultrasonic image) on which the puncturing needle emphasis, the neural emphasis, and the vascular emphasis are superimposed in FIG. 14B. Then, the state of success or failure of the femoral nerve block is reported to the emergency physician, and an improvement measure is advised as necessary.

### Fourth Embodiment

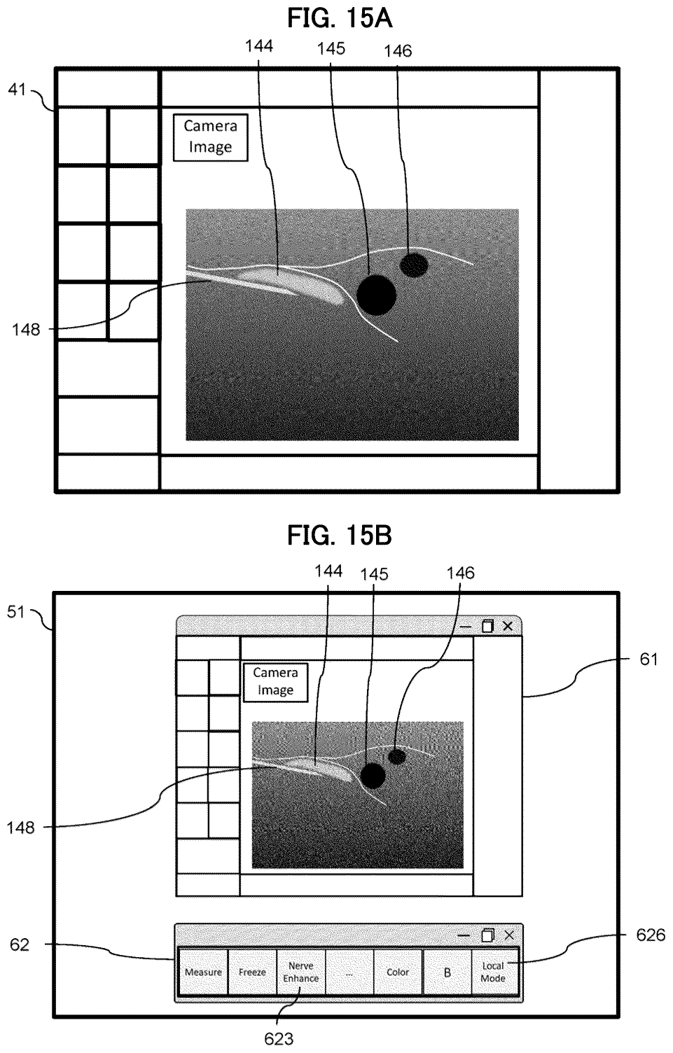

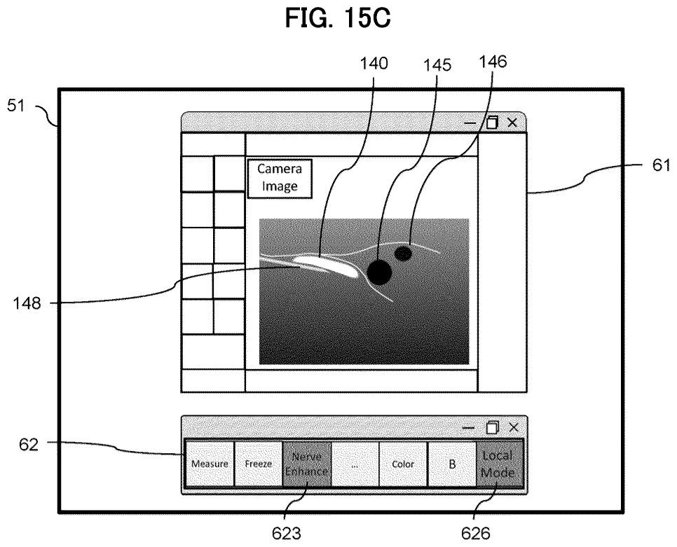

Next, an example of performing femoral nerve block on a femur fracture patient using the embodiment of the ultrasonic image diagnostic system 1 of the above-described embodiment will be described with reference to FIGS. 15A to 15C. In this example, the ultrasonic diagnostic apparatus operated by the emergency doctor displays the B-mode image, and the information terminal operated by the anesthesiologist at the remote place instructs the independent display to display the B-mode image on which the nerve enhancement data is superimposed.

Specifically, the emergency physician first causes the ultrasonic diagnostic apparatus 2 to display the B-mode image on which the nerve enhancement data is superimposed, and checks the position of the femoral nerve. Thereafter, as illustrated in FIG. 15A, the emergency physician performs femoral nerve block in a state where the B-mode image on which the nerve-emphasized image is not superimposed is displayed on the ultrasonic diagnostic apparatus 2. At this time, the information terminal 3 displays the B-mode image of FIG. 15B synchronized with FIG. 15A. The specialist instructs the ultrasonic diagnostic apparatus 2 to perform independent display so as to display the B-mode image in a state in which the nerve enhancement function is enabled, in order to confirm that there is no misrecognition or observation omission in a site having a particularly large difference from typical nerve travel.

The ultrasonic diagnostic apparatus 2 transmits the nerve-emphasized image and the B-mode image to the information terminal 3 on the basis of the independent display instruction from the information terminal 3, and the information terminal 3 displays, on the display part 51, the B-mode image on which the nerve-emphasized image is superimposed (FIG. 15C). The specialist observes the B-mode image on which the nerve enhancement data is superimposed and which is displayed on the display part 51 of the information terminal 3, and checks that there is no misrecognition or observation omission regarding the running of the nerve.

Hereinafter, the detailed description will be given with reference to the flowchart of FIG. 2.

In step S21, the emergency physician activates the ultrasonic diagnostic apparatus 2 with the ultrasonic probe 42 and the camera 45 connected to each other. On the other hand, a specialist activates a PC (information terminal 3) including the camera 55 and a touch-operable monitor (input section 53+display part 51) from a remote hospital, and remotely connects the PC to the ultrasonic diagnostic apparatus 2.

In step S22, after an emergency doctor registers information about a patient in the DB, the ultrasonic diagnostic apparatus 2 selects the B mode as the imaging mode and starts imaging of an ultrasonic image. Then, the ultrasonic diagnostic apparatus 2 performs a pre-scan of the vicinity of the surgical field in a state where the nerve enhancement function is enabled. After the completion of the pre-scanning, the ultrasonic diagnostic apparatus 2 disables the nerve enhancement function and displays only the normal B-mode image.

The emergency physician starts puncturing while adjusting the positions of the ultrasonic probe and the puncture needle. In step S23, the ultrasonic diagnostic apparatus 2 generates a normal B-mode image as a first ultrasonic image based on an ultrasonic signal acquired by scanning the body surface of the patient using the connected linear probe, and displays the image on the display part 41 of the ultrasonic diagnostic apparatus 2 as shown in FIG. 15A.

On the display part 41 of the ultrasonic diagnostic apparatus 2, the puncture needles 148, femoral nerves 144, femoral arteries 145, and femoral veins 146 are displayed on the B-mode image, as shown in FIG. 15A. The emergency physician advances the needle toward the femoral nerve, which is a target, while checking the positions of the puncture needle and the femoral artery and vein. Then, the emergency physician injects a drug at the time point when the puncture needle reaches the affected part.

In step S24, since a remote operation from the information terminal 3 is not performed in the present example, the processes of step S25 and step S26 are not performed, and the process proceeds to step S27.

In FIG. 15B, the medical specialist taps the “Local Mode” button 626 and the “Nerve Enhance” button 623 of the remote operation floating window 62. Here, “Local Mode” button 626 is an operation button for executing an instruction related to independent display. The “Nerve Enhance” button 623 instructs the start/end of the neural enhancement mode. The specialist taps the “Nerve Enhance” button 623 in order to give an instruction to generate and transmit a nerve-enhanced dataset and a B-mode image.

Accordingly, in step S27, the ultrasonic image diagnostic system 1 determines that the independent display instruction has been received, and the process proceeds to step S28.

In step S28, the ultrasonic diagnostic apparatus 2 generates a B-mode ultrasonic image on which the nerve-emphasized image 140 is superimposed and transmits the B-mode ultrasonic image to the information terminal 3, and the information terminal 3 displays the transmitted ultrasonic image as a second ultrasonic image on the display part 51, as shown in FIG. 15C.

The medical specialist observes the nerve-emphasized image on the B-mode image at a site where the difference from the typical neural transit is particularly large by the ultrasonic image of FIG. 15C, and confirms that there is no misrecognition or observation omission regarding the neural transit. The medical specialist taps the “Local Mode” button 626 after the confirmation, and notifies the ultrasonic diagnostic apparatus 2 of an independent display cancellation instruction.

In step S29, in response to reception of the independent display cancellation instruction from the information terminal 3, the ultrasonic diagnostic apparatus 2 returns to step S23, and the information terminal 3 returns the display on the display part 51 to the state of the synchronous display of the B-mode image in FIG. 15B. Then, the state of success or failure of the femoral nerve block is reported to the emergency physician, and an improvement measure is advised as necessary.

### Fifth Embodiment

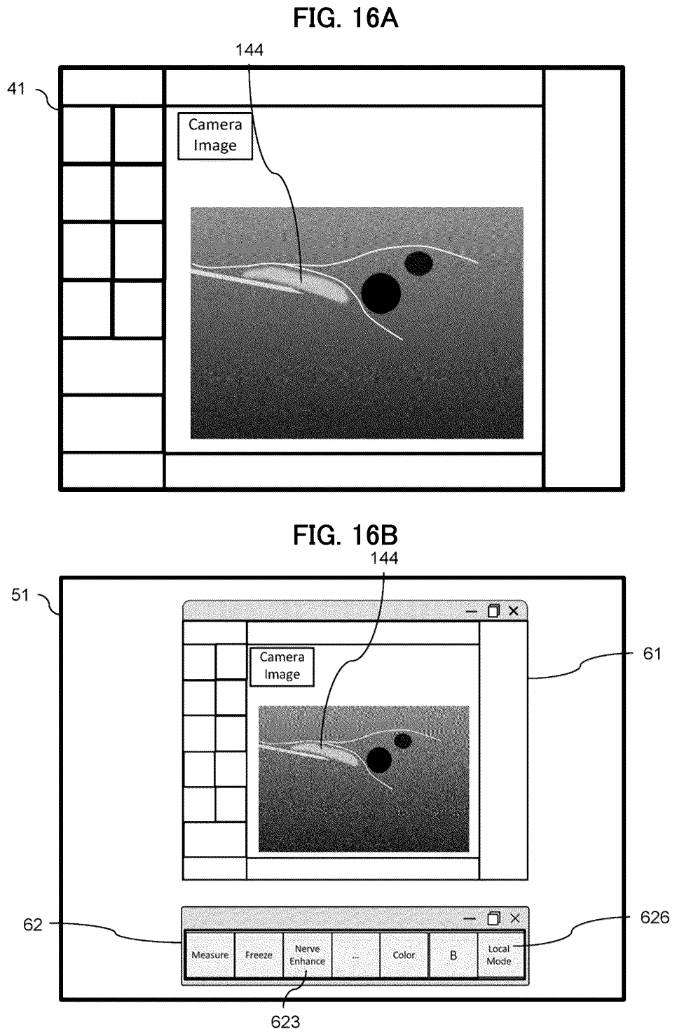

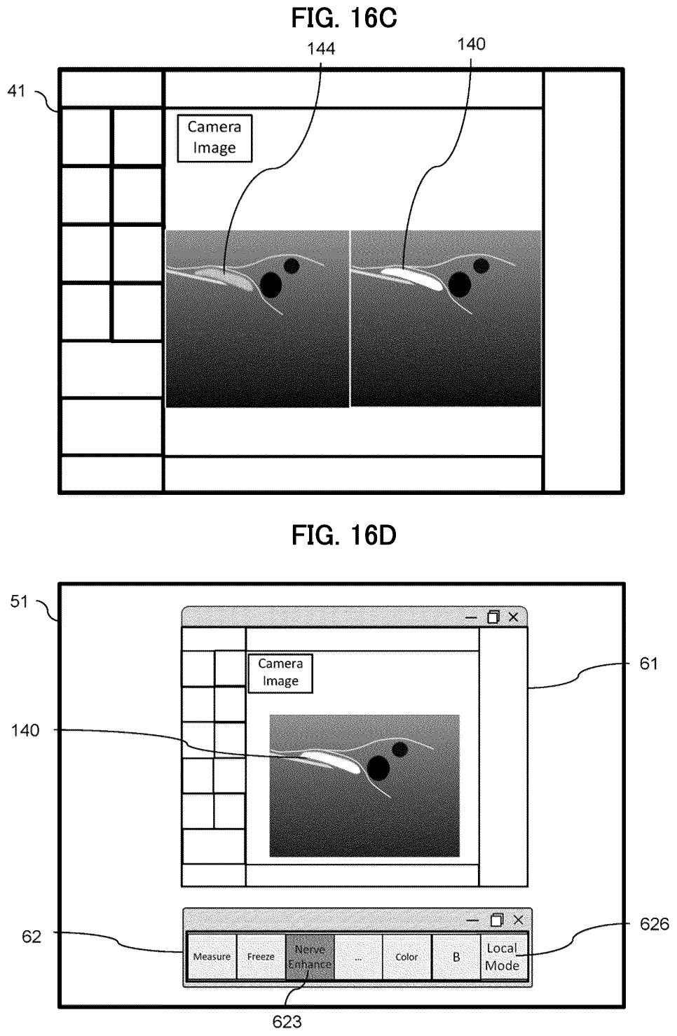

Next, a description will be given, with reference to FIGS. 16A to 16D, of an operation performed when an orthopedic doctor uses the embodiment of the ultrasonic image diagnostic system 1 to send a message regarding the condition of a patient and a therapeutic result to a physical therapist. At this time, it is assumed that the orthopedic surgeon uses the ultrasonic diagnostic apparatus 2 in the examination room and the physical therapist uses the information terminal 3 in the rehabilitation room.

Specifically, first, as illustrated in FIG. 16A, the ultrasonic diagnostic apparatus 2 used by the orthopedic surgeon displays a B-mode image including the femoral nerve 144. Then, the information terminal 3 used by the physical therapist displays a B-mode image synchronized with the ultrasonic image of the ultrasonic diagnostic apparatus 2 as a first ultrasonic image, as shown in FIG. 16B.

Thereafter, the physical therapist presses the independent display instruction button 626 and the nerve enhancement button 623 on the information terminal 3, thereby instructing the ultrasonic diagnostic apparatus 2 to generate and transmit a B-mode image in which nerve enhancement display is enabled. As illustrated in the FIG. 16C, the ultrasonic diagnostic apparatus 2 causes its own display part 41 to display a normal B-mode image and a B-mode image in which the neural highlighting (the neural highlighting 140) is enabled, side by side. Further, the ultrasonic diagnostic apparatus 2 generates a B-mode image in which the neural highlighting is enabled and transfers the B-mode image to the information terminal 3, and the information terminal 3 displays the B-mode image on the display part 51 as shown in FIG. 16D. Thus, an orthopedic surgeon who operates the ultrasonic diagnostic apparatus 2 and a physical therapist who operates the information terminal 3 can consult patient's clinical condition, treatment result, and rehabilitation plan while acquiring an ultrasonic image with display setting suitable for each observation purpose and environment.

The following operation is performed in the ultrasonic image diagnostic system 1 of the present example. That is, it is assumed that the permission request regarding the linkage function between the information terminal 3 and the ultrasonic diagnostic apparatus 2 as shown in the following (I) to (iii) is performed from the information terminal 3 to the ultrasonic diagnostic apparatus 2, and the ultrasonic diagnostic apparatus 2 permits the information terminal 3.
(i) A function of sharing an ultrasonic image and a camera image between the display part 41 of the ultrasonic diagnostic apparatus 2 and the display part 51 of the information terminal 3 by transmitting the ultrasonic image live from the ultrasonic diagnostic apparatus 2 to the information terminal 3 or transmitting the camera images of the ultrasonic diagnostic apparatus 2 and the information terminal 3 live from each other.
(ii) A function of transmitting a remote operation command from the information terminal 3 to the ultrasonic diagnostic apparatus 2.
(iii) A function of performing voice communication via the microphone connected to the ultrasonic diagnostic apparatus 2 and the information terminal 3.

Further, the orthopedic surgeon registers information on the patient in the patient database of the ultrasonic diagnostic apparatus 2 and selects the B mode as the imaging mode.

Hereinafter, detailed description will be given with reference to the flowchart of FIG. 2.

In step S21, the orthopedic surgeon starts the ultrasonic diagnostic apparatus 2 in a state where the ultrasonic probe 42 and the camera 45 are connected. On the other hand, the physical therapist activates a portable information terminal (information terminal 3) including the camera 55 and a touch-operable monitor (input section 53+display part 51) from the rehabilitation room, and remotely connects the portable information terminal to the ultrasonic diagnostic apparatus 2.

In step S22, after an orthopedic surgeon registers information on a patient in a DB, the ultrasonic diagnostic apparatus 2 selects preset and an imaging mode (B mode) and starts imaging of an ultrasonic image.

The orthopedic surgeon explains the pathological condition of the patient and the treatment result (the state of the hydroreleased nerve) to the physical therapist while capturing an ultrasonic image while moving the ultrasonic probe 42 between the proximal side and the distal side.

In response to this, in step S23, the ultrasonic diagnostic apparatus 2 generates a B-mode image as a first ultrasonic image based on the ultrasonic signal of the ultrasonic probe 42, and displays the ultrasonic image on the display part 41 as illustrated in FIG. 16A. Furthermore, the ultrasonic diagnostic apparatus 2 transmits the generated B-mode image to the information terminal 3, and the information terminal 3 displays the received B-mode image as an ultrasonic image on the display part 51 for sharing, as illustrated in FIG. 16B.

In step S24, since a remote operation from the information terminal 3 is not performed in the present example, the processes of step S25 and step S26 are not performed, and the process proceeds to step S27.

In the screen of the FIG. 16B, the physiotherapist taps the “Local Mode” button 626 and the “Nerve Enhance” button 623 of the remote operation floating window 62 in order to confirm the site. Here, the “Local Mode” button 626 is an operation button for executing an instruction related to independent display. The “Nerve Enhance” button 623 is an operation button for giving an instruction to start the neural enhancement mode. That is, the physical therapist instructs the ultrasonic diagnostic apparatus 2 to generate and transmit a B-mode image in which nerve highlighting is enabled.

Accordingly, in step S27, the ultrasonic image diagnostic system 1 determines that the independent display instruction has been received, and the process proceeds to step S28.

In step S28, the ultrasonic diagnostic apparatus 2 generates a B-mode image on which the neuron emphasis data 140 emphasizing the neuron region is superimposed, and transmits the B-mode image as an ultrasonic image to the information terminal 3. Next, as illustrated in FIG. 16D, the information terminal 3 displays the transmitted ultrasonic image on the display part 51. At this time, as illustrated in FIG. 16C, the ultrasonic diagnostic apparatus 2 displays the “B-mode image on which the nerve-enhancing data 140 is not superimposed” and the “B-mode image on which the nerve-enhancing data 140 is superimposed” side by side.

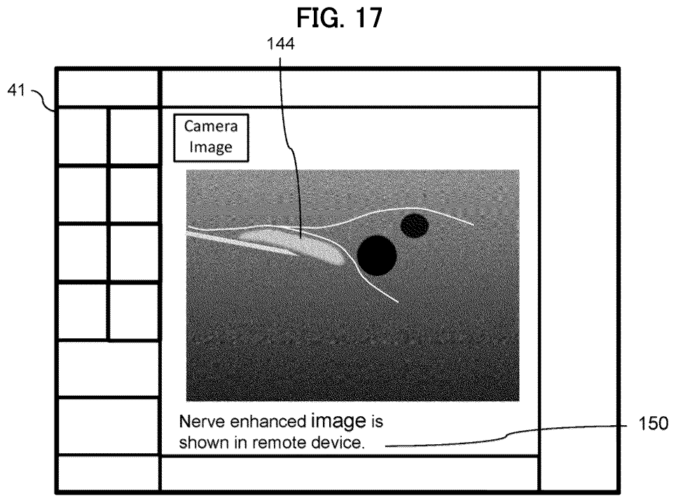

Furthermore, instead of the juxtaposition display, the ultrasonic diagnostic apparatus 2 may display the “B-mode image on which the nerve enhancement data 140 is not superimposed” (the B-mode image in which the femoral nerve 144 is displayed) in FIG. 17, and display a message indicating that the nerve enhancement image is being displayed on the information terminal 3. For example, “Nerve enhanced image is shown in remote device.” 150 is displayed. Note that a B-mode image in which nerve emphasis display is disabled may be displayed in another tab.

When the physical therapist confirms the advice regarding the condition of the patient and the treatment result, the physical therapist informs the orthopedic surgeon of a rehabilitation plan suitable for the condition of the patient and the treatment result. Then, the physical therapist taps the “Local Mode” button 626 to notify the ultrasonic diagnostic apparatus 2 of an independent display cancellation instruction.

In step S29, in response to reception of the independent display cancellation instruction from the information terminal 3, the ultrasonic diagnostic apparatus 2 returns to step S23, and the information terminal 3 returns the display on the display part 51 to the state of the synchronous display of the B-mode image in FIG. 16B.

Although various embodiment and modification example have been described in detail, the independent display and the instruction related to the release of the independent display illustrated in the present invention may be implemented by executing an operation of designating a predetermined mode or preset on the display part of the ultrasonic diagnostic apparatus 2 or the information terminal 3.

Further, the present invention is not limited to the above-described embodiment, and includes various modification examples. The above-described embodiment has been described in detail for easy understanding of the present invention, and the present invention is not necessarily limited to those having all the configurations described above. Furthermore, part of the configuration of an embodiment can be replaced with the configuration of another embodiment, and the configuration of another embodiment can be added to the configuration of an embodiment.

### DESCRIPTION OF REFERENCE NUMERALS

1 ULTRASONIC IMAGE DIAGNOSTIC SYSTEM
2 ULTRASONIC DIAGNOSTIC APPARATUS
21 CONTROLLER
22 DISPLAY PROCESSING SECTION
23 COMMUNICATION SECTION
24 SIGNAL PROCESSOR (IMAGE GENERATION SECTION)
3 INFORMATION TERMINALS
31 CONTROLLER
32 DISPLAY PROCESSING SECTION
33 COMMUNICATION SECTION
41 DISPLAY PART
42 ULTRASONIC PROBE (ULTRASONIC PROBE)
43 INPUT SECTION
51 DISPLAY PART
53 INPUT SECTION

What is claimed is:

1. An ultrasonic image diagnostic system comprising:
an ultrasonic diagnostic apparatus which is adapted to generate a first ultrasonic image based on a first ultrasonic image data obtained by transmitting and receiving ultrasonic signals using an ultrasonic probe, wherein the ultrasonic diagnostic apparatus has a display part for displaying the first ultrasonic image, and
an information terminal having an input part and a display part, wherein the ultrasonic diagnostic apparatus and the information terminal are connected via a network, wherein
the ultrasonic diagnostic apparatus generates a second ultrasonic image from a second ultrasonic image data different from the first ultrasonic image data in response to an instruction sent from the input part of the information terminal to the ultrasonic diagnostic apparatus, and
the display part of the information terminal is adapted to display the second ultrasonic image independently of the first ultrasonic image displayed on the ultrasonic diagnostic apparatus.

2. The ultrasonic image diagnostic system according to claim 1, wherein the display part of the information terminal changes from a state in which the first ultrasonic image is displayed to a state in which the second ultrasonic image is independently displayed in response to the instruction.

3. The ultrasonic diagnostic apparatus according to claim 2, wherein the information terminal includes an instruction unit which is adapted to give an instruction from the input part to the ultrasonic diagnostic apparatus, and wherein
the instruction unit is an independent display instruction unit which is adapted to allow the display part of the information terminal to independently display the second ultrasonic image.

4. The ultrasonic diagnostic apparatus according to claim 2, wherein the ultrasonic diagnostic apparatus includes an independent display instruction unit for instructing the display part of the information terminal to independently display the second ultrasonic image.

5. The ultrasonic image diagnostic system according to claim 1, wherein the display part of the information terminal is adapted to change from a state in which the second ultrasonic image is independently displayed to a state in which the first ultrasonic image is displayed.

6. The ultrasonic image diagnostic system according to claim 5, wherein the display part of the information terminal includes an independent display cancelling unit that is adapted to allow the display part of the information terminal to change from the state in which the second ultrasonic image is independently displayed to the state in which the first ultrasonic image is displayed.

7. The ultrasonic image diagnostic system according to claim 1, wherein the instruction is an instruction for the ultrasonic diagnostic apparatus to specify a predetermined mode or a preset.

8. A computer readable nonvolatile storage medium storing a computer program, the computer program, when executed by a processor of an ultrasonic diagnostic apparatus including an ultrasonic probe and a display part, causing the processor to perform
generating first ultrasonic image data from an ultrasonic signal transmitted and received using the ultrasonic probe;
displaying a first ultrasonic image based on the first ultrasonic image data on the display part;
transmitting the first ultrasonic image data to an information processing apparatus;
independently generating second ultrasonic image data different from the first ultrasonic image data based on an instruction from the information processing apparatus; and
transmitting the second ultrasonic image data to the information processing apparatus.

9. A computer readable nonvolatile storage medium storing a computer program, the computer program, when executed by a processor of an information processing apparatus including an input section and a display part, causing the processor to perform
receiving first ultrasonic image data from an ultrasonic diagnostic apparatus which displays a first ultrasonic image;
displaying a first ultrasonic image based on the first ultrasonic image data on the display part;
instructing the ultrasonic diagnostic apparatus by an input from the input section;
receiving second ultrasonic image data different from the first ultrasonic image data from the ultrasonic diagnostic apparatus; and
displaying a second ultrasonic image based on the second ultrasonic image data on the display part.

10. An ultrasonic diagnostic apparatus comprising:
an image data generation section generating first ultrasonic image data and/or second ultrasonic image data on a basis of ultrasonic signals transmitted and received by using an ultrasonic probe;
a display part displaying an ultrasonic image generated on the basis of the first ultrasonic image data or the second ultrasonic image data; and
a communication section transmitting the first ultrasonic image data or the second ultrasonic image data to an information processing apparatus and receiving an instruction from the information processing apparatus, wherein
the image data generation section independently generates the second ultrasonic image data different from the first ultrasonic image data according to the instruction received by the communication section when the first ultrasonic image is displayed on the display part, and
the communication section transmits the second ultrasonic image data to the information processing apparatus.

11. An ultrasonic image diagnostic method performed by an ultrasonic diagnostic apparatus including a processor and a display part, the method comprising causing the processor of the ultrasonic diagnostic apparatus to
generate first ultrasonic image data from ultrasonic signals transmitted and received using an ultrasonic probe;
display a first ultrasonic image based on the first ultrasonic image data on the display part;
transmit the first ultrasonic image data to an information processing apparatus;
independently generate second ultrasonic image data different from the first ultrasonic image data based on an instruction from the information processing apparatus; and
transmit the second ultrasonic image data to the information processing apparatus.
